(19) United States
(12) Patent Application Publication
(10) Pub. No.: US 2021/0035037 A1
(43) Pub. Date: Feb. 4, 2021

(54) SERVER, SYSTEM, AND INFORMATION PROCESSING METHOD

(71) Applicants: TOYOTA JIDOSHA KABUSHIKI KAISHA, Toyota-shi (JP); DENSO CORPORATION, Kariya-city (JP)

(72) Inventors: Yukari SUZUKI, Setagaya-ku (JP); Naoki YAMADA, Toyota-shi (JP); Toshihiro ARAI, Nagoya-shi (JP); Kuniaki JINNAI, Nagoya-shi (JP); Keiichi UNO, Chita-gun (JP)

(73) Assignees: TOYOTA JIDOSHA KABUSHIKI KAISHA, Toyota-shi (JP); DENSO CORPORATION, Kariya-city (JP)

(21) Appl. No.: 16/943,039

(22) Filed: Jul. 30, 2020

(30) Foreign Application Priority Data
Jul. 30, 2019 (JP) 2019-140172

(51) Int. Cl. G06Q 10/06; H04L 29/08

(52) U.S. Cl. CPC G06Q 10/06311; H04L 67/12

(57) ABSTRACT

Provided is a system including a server, a terminal apparatus of a user, and a vehicle configured to provide a transportation service for taking the user to a hospital. The server includes an acquiring means configured to acquire information about a state of health of the user, a memory configured to store information about a physician, and a controller configured to perform matching by collating the information about the state of health of the user with the information about the physician, sets a consultation date and time with the physician, and generates a dispatch schedule of a vehicle on a basis of the consultation date and time. The vehicle is driven in accordance with the dispatch schedule.

### CROSS-REFERENCES TO RELATED APPLICATIONS

The present application claims the benefit of Japanese Patent Application No. 2019140172, filed in the Japan Patent Office on Jul. 30, 2019, which is incorporated by reference herein in its entirety.

### TECHNICAL FIELD

The present disclosure relates to a server, a system, and an information processing method, and more particularly, to a server, a system, and an information processing method for providing a transportation service to take a user to a hospital.

### BACKGROUND

In recent years, there has been a demand for medical systems to allow a patient to select an appropriate physician with regard to the patient's own disease. To meet such needs, PTL 1 proposes a medical system comprising a patient condition acquisition means that acquires patient condition information identifying the condition of a patient, a physician information storing means that stores physician information regarding a physician, and a matching means that matches the patient to the physician based on the patient condition information acquired by the patient condition acquiring means and the physician information stored in the physician information storing means.

### CITATION LIST

### Patent Literature

PTL 1: WO2017158754

### SUMMARY

However, even if an elderly person or a disabled person can select an appropriate physician or hospital with regard to the person's own disease, the person may not be able to visit the hospital easily in such cases as where the hospital is far away, or where no appropriate means of transportation is available. The person, when visiting a hospital, needs to take such procedures as for making an appointment with the hospital and for reserving or scheduling a means of transportation, which may be found as burdensome particularly for elderly and disabled.

Consequently, it would be helpful to provide a server, a system, and an information processing method for allowing selection of a physician appropriate for a disease of a user, while furthermore improving the convenience of making an appointment for a consultation with the physician and visiting the hospital.

A server according to an embodiment of the present disclosure provides a transportation service to take a user to a hospital, the server comprising an acquiring means configured to acquire information about a state of health of the user, a memory configured to store information about a physician, and a controller configured to perform matching by collating the information about the state of health of the user with the information about the physician, sets a date and time of a consultation with the physician, and generates a dispatch schedule of a vehicle based on the consultation date and time.

A system according to an embodiment of the present disclosure includes the server, a terminal apparatus of the user, and a vehicle configured to provides a transportation service to take the user to a hospital. The terminal apparatus receives the set desired consultation date and time and the dispatch schedule, and the vehicle is driven in accordance with the dispatch schedule.

Also, an information processing method according to an embodiment of the present disclosure provides a transportation service to take a user to a hospital, the method including acquiring information about a state of health of the user, reading out information about a physician, performing matching by collating the information about the state of health of the user with the information about the physician, and setting a date and time of a consultation with the physician, and generating a dispatch schedule of a vehicle, based on the consultation date and time.

The server, system, and information processing method of the present disclosure allow for selecting a physician appropriate for a disease of a user while furthermore improving the convenience of making an appointment for a consultation with the physician and visiting the hospital.

### BRIEF DESCRIPTION OF THE DRAWINGS

In the accompanying drawings:

FIG. 1 is a diagram illustrating an exemplary configuration of a system according to an embodiment;

FIG. 2 is a diagram illustrating an exemplary configuration of a server according to an embodiment;

FIG. 3 is a diagram illustrating an exemplary configuration of a physician database;

FIG. 4 is a flowchart illustrating an example of an information processing method by the server;

FIG. 5 is a conceptual diagram illustrating an example of screen transitions on a terminal apparatus;

FIG. 6 is a diagram illustrating an exemplary configuration of a vehicle according to an embodiment; and

FIG. 7 is a sequence diagram illustrating an example of overall operations by a system according to an embodiment.

### DETAILED DESCRIPTION

Hereinafter, an embodiment for carrying out the present disclosure will be described with reference to the drawings. In the drawings, identical or similar components are denoted with the same signs.

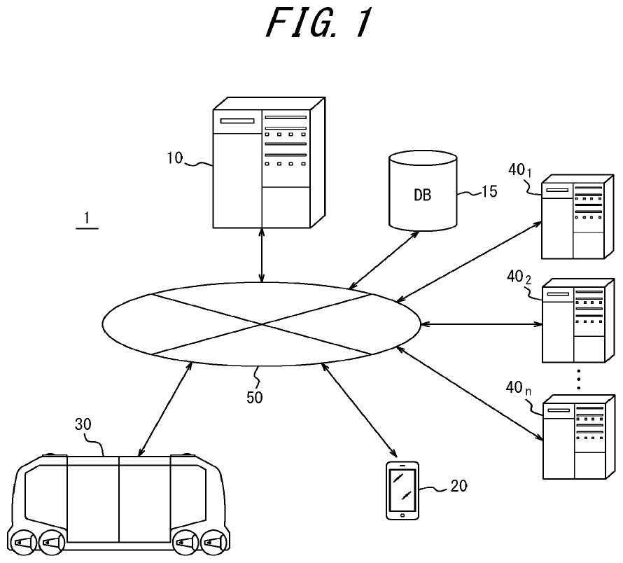

FIG. 1 is a diagram illustrating an exemplary configuration of a system for providing a transportation service to take a user to a hospital, according to an embodiment of the present disclosure. A system 1 according to the present embodiment includes a server 10, a terminal apparatus 20 of a user, and a vehicle 30. The server 10, the terminal apparatus 20 of a user, and the vehicle 30 are connected by a network 50 of any type to allow for information communication. Additionally, in the system 1, a database (DB) 15 and external servers 40 (40₁ to 40ₙ) of hospitals or the like are connected to the network 50 as necessary. Although one terminal apparatus 20 and one vehicle 30 are illustrated in FIG. 1, a plurality of each may also be connected to the network 50.

To give an overview, the present disclosure is a server, a system, and an information processing method for providing a transportation service to take a user to a hospital. The server 10 performs matching by collating information about the user's state of health (such as a condition) with information about physicians, and sets a physician and a consultation date and time appropriate for the user's state of health. Additionally, the server 10 generates a dispatch schedule based on the consultation date and time. Thereafter, the vehicle 30 is driven to take the user to the hospital based on the dispatch schedule.

The server 10 acquires information about the user's state of health, from the terminal apparatus 20 of the user. The server 10 may also acquire the information about the user's state of health by any other means, such as by obtaining information from the database 15 storing information about the user's state of health, for example. Herein, the user refers to a user of the system 1, and is a person who desires transportation service to an appropriate hospital. Also, the information about the user's state of health includes information such as the condition of the user, the name of a disease of the user, or a health checkup result for the user.

The server 10 performs matching by collating the information about the user's state of health with information about physicians, selects a physician suited to the user's state of health (such as a condition), and presents the selection to the user. The server 10 also makes a consultation appointment for the user based on the physician's appointment schedule and the user's desired consultation date and time. Additionally, the server 10 generates a dispatch schedule of the vehicle 30 based on the consultation appointment date and time. The configuration and operation of the server 10 will be described in detail later.

The database 15 stores information of any type related to the system 1. For example, the database 15 may be a public database storing information such as the career histories and achievements of physicians, or a database of any type storing information such as user ratings and reviews of physicians. The database 15 may also store information about the user's state of health (for example, a health checkup result).

The terminal apparatus 20 is a communication apparatus used by the user, and may be a device such as a mobile phone, a smartphone, a smart speaker, or a personal computer (PC) for example. The terminal apparatus 20 is not limited to the above, and may be any type of apparatus used by the user.

The terminal apparatus 20 is capable of transmitting information about the user's state of health to the server 10. The terminal apparatus 20 is also capable of receiving information about a physician appropriate for the condition of the user, a consultation appointment date and time, a dispatch schedule of the vehicle 30, and the like from the server 10. The dispatch schedule is information about the schedule for dispatching the vehicle 30 to provide a transportation service to the hospital, and includes information such as destinations, a driving route, a scheduled arrival time at each point, parking locations, and parking times.

The vehicle 30 provides the transportation service to take the user to the hospital. Specifically, the vehicle 30 picks up the user and drives from the user's house to the hospital where a consultation is scheduled, based on the dispatch schedule set by the server 10. Furthermore, the vehicle 30 may also drive the user from the hospital back home as necessary. The vehicle 30 may drive a specific user to a single hospital, but may also pick up a plurality of users and stop at a plurality of hospitals in sequence. A plurality of vehicles 30 may also exist, and it is desirable to adjust factors such as the driving course as well as the size and number of vehicles in correspondence with the user(s). As described later, the vehicle 30 may also perform autonomous driving or follow-up driving.

The external servers 40 (40₁ to 40ₙ) are servers owned by medical institutions such as hospitals, and may be connected to the system 1. The external servers 40 provide physician appointment schedules to the server 10. The external servers 40 also receive the user's consultation appointment from the server 10.

The network 50 is a communication network of any type allowing the server 10, the terminal apparatus 20, the vehicle 30, and the external servers 40 to communicate with each other. The network 50 may be a wireless or wired transmission channel, and may be a communication network such as the Internet. For example, the network 50 in the present embodiment may include an ad hoc network, a metropolitan area network (MAN), a cellular network, a wireless personal area network (WPAN), the public switched telephone network (PSTN), a terrestrial wireless network, an optical network, some other network, or a combination of any of the above. A wireless network includes components such as an access point (for example, a Wi-Fi access point) and femtocells. Additionally, wireless communication equipment can be connected to a wireless network using Wi-Fi® (Wi-Fi is a registered trademark in Japan, other countries, or both), Bluetooth® (Bluetooth is a registered trademark in Japan, other countries, or both), cellular communication technology, or some other wireless technology and technology standard.

According to the system 1, the server 10 selects and sets a consultation appointment with a physician appropriate for the condition of the user, and the vehicle 30 provides a service of picking up and taking the user from the user's home to the hospital based on the dispatch schedule. With this arrangement, the user is able to make a consultation appointment and visit the hospital easily, and receive treatment from an appropriate physician.

In the embodiment above, the database 15 is described as being connected to the network 50 of the system 1; however, in cases where the information stored in the database 15 is not used, the database 15 may be omitted. The external servers 40 of hospitals and the like are described as being connected to the network 50, but if the system 1 can obtain information about the hospitals by some other means, the external servers 40 may be omitted.

Next, the configuration and operation (information processing method) of the server 10 will be described in detail.

(Server Configuration)

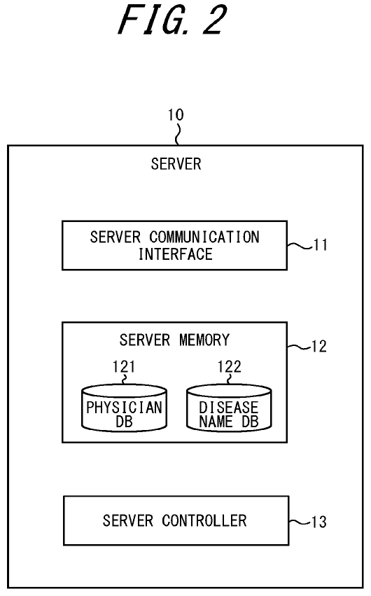

FIG. 2 is a diagram illustrating an exemplary configuration of the server 10 according to an embodiment. The server 10 includes a server communication interface 11, server memory 12, and a server controller 13.

The server communication interface (also simply referred to as the communication interface) 11 includes a communication module that connects to the network 50. The communication module supports mobile communication standards such as 4th Generation (4G) and 5th Generation (5G), a wired local area network (LAN) standard, or a wireless LAN standard for example, but is not limited to the above, and may support any type of communication standard. In the present embodiment, the server 10 is connected to the network 50 through the server communication interface 11. The server communication interface 11 receives (acquires) information (such as information related to the user's state of health, for example) transmitted from the database 15, the terminal apparatus 20 of the user, or the external servers 40 over the network 50. The server communication interface 11 is also capable of transmitting information (such as the dispatch schedule and the consultation appointment, for example) to the terminal apparatus 20 of the user, the vehicle 30, or the external servers 40 over the network 50. In the present embodiment, the server communication interface 11 is an acquiring means that acquires information about the user's state of health.

The server memory 12 (also simply referred to as the memory) 12 includes semiconductor memory or magnetic memory. The server memory 12 may function as main memory, auxiliary memory, or cache memory, for example. The server memory 12 stores information of any type used in the operation of the server 10. For example, the server memory 12 stores information such as a system program, an application program, and a management database. It may also be possible to update the information stored in the server memory 12 with information acquired from the network 50 through the server communication interface 11 for example.

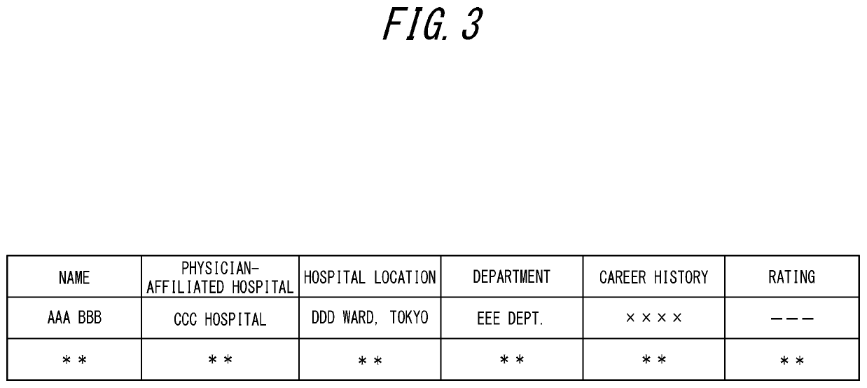

In the present embodiment, the server memory 12 includes a physician database (DB) 121 and a disease name database (DB) 122. Information about physicians that can be recommended to the user in the system 1 is registered in the physician database 121. For example, as illustrated in FIG. 3, information such as the name of a physician, the physician-affiliated hospital, the location of the hospital, the department, a career history, and a rating are registered in the physician database 121. The information about a physician may be data that the server 10 stores separately in the physician database 121, or data that the server 10 acquires from the other database 15 or the external servers 40 and stores. Herein, the career history includes information such as a history of the physician's career and cases that the physician has treated so far. Also, the rating may be a rating determined by the server 10 based on the career history and achievements of the physician, and may also include information such as an internal rating by the physician-affiliated hospital or external user rating and review information. The items in the physician database 121 can be set as necessary. The disease name database 122 stores information related to diseases and their symptoms. In addition, the server memory 12 may also store the consultation appointment time for the user and the dispatch schedule of the vehicle 30 that picks up the user.

The server controller (also simply referred to as the controller) 13 includes one or more processors. In the present embodiment, “processor” refers to, but is not limited to, a general-purpose processor or a special-purpose processor specialized for a specific process. The server controller 13 controls the operation of the server 10 as a whole. For example, the server controller 13 controls the server 10 based on programs such as the system program and the application program stored in the server memory 12.

In the present embodiment, the server controller 13 references the physician database 121 and the disease name database 122, matches the information about the user's state of health with the information about physicians, and selects, as a candidate physician, a physician providing a highly reliable treatment with respect to the condition of the user. Additionally, the server controller 13 makes a consultation appointment with the physician based on at least one of the physician's appointment schedule and the user's desired consultation date and time. Furthermore, the server controller 13 decides a dispatch schedule of the vehicle 30 for providing the transportation service to the user based on the consultation date and time in the appointment, and transmits the dispatch schedule to the vehicle 30 and the terminal apparatus 20 through the server communication interface 11.

Also, the server controller 13 is capable of receiving driving information about the vehicle 30 (such as position information and the traveling speed of the vehicle 30) from the vehicle 30 through the server communication interface 11. The server controller 13 stores the driving information in the server memory 12, and may correct the dispatch schedule of the vehicle 30 as necessary based on the stored information. The server controller 13 may also transmit update information for updating the dispatch schedule of the vehicle 30, the system program, the application program, or the like to the vehicle 30 through the server communication interface 11.

(Information Processing Method by Server)

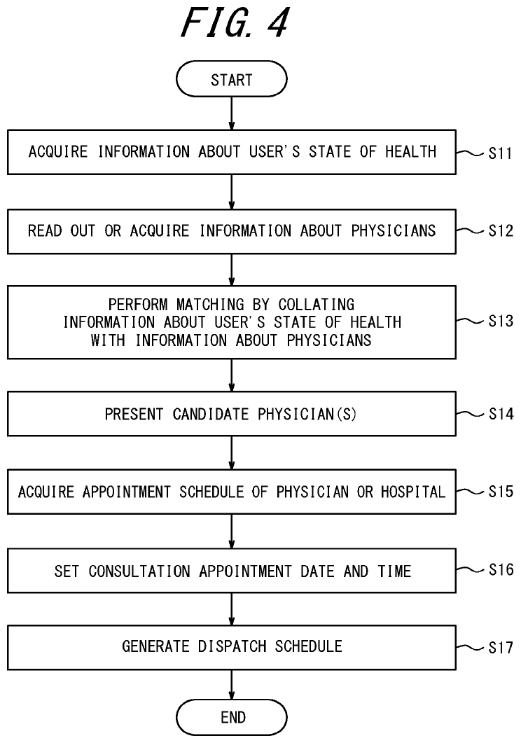

FIG. 4 is a flowchart illustrating an example of an information processing method by the server 10. The server 10 executes the following steps S11 to S17 to make a consultation appointment and provide the transportation service to the hospital where an optimal physician for the user is present.

Step S11: The server 10 acquires information about the user's state of health from the terminal apparatus 20 of the user or the other database 15. For example, the server 10 may receive information about the user's state of health (for example, the condition or disease) transmitted from the terminal apparatus 20 used by the user, or utilize result data from a health checkup of the user stored in the other database 15 as the information about the user's state of health. The server 10 stores the acquired information about the user's state of health in the server memory 12.

Step S12: The server 10 reads out registered information about physicians from the physician database 121 of the server memory 12. Alternatively, the server 10 may acquire the information about physicians over the network 50 from the external servers 40 (40₁ to 40ₙ) owned by hospitals (or medical institutions).

Step S13: The server 10 performs matching by collating the information about the user's state of health with the information about physicians, and selects an optimal physician for the user's state of health. For example, the server 10 infers a disease and also determines a corresponding department from the information about the user's state of health (for example, the condition) based on information about the conditions of diseases stored in the disease name database 122. Furthermore, the server 10 matches the determined disease or the information about the user's state of health with the information about physicians read out from the physician database 121 or acquired from the external servers 40, and selects a physician providing highly reliable treatment with respect to the condition of the user as a candidate physician. The reliability of treatment with respect to a condition can be quantified and determined by applying predetermined coefficients to the career histories of physicians, their experience dealing with similar cases, and ratings of the physicians, and furthermore may also be determined by some other technique. The number of candidate physicians is not limited to one, and a plurality of physicians may be selected in order of highest reliability. When selecting the physician(s), locality may also be considered as necessary, and the selection can also be limited to physicians residing in the same prefecture or an adjacent prefecture to the user's address.

Step S14: The server 10 transmits information about the physician(s) providing highly reliable treatment with respect to the condition of the user obtained as a result of the matching to the terminal apparatus 20, and presents the candidate physician(s) to the user. The information about the candidate physician(s) to present includes the name of each candidate physician as well as related information about each physician (such as the physician-affiliated hospital and a career history). Information such as the inferred disease and the department may also be transmitted at the same time.

Step S15: For a physician selected (chosen) or approved by the user, the server 10 acquires the appointment schedule of the physician (which in some cases also includes the appointment schedule of the physician-affiliated hospital of the physician) from the external server 40 of the physician-affiliated hospital of the physician. Thereafter, the server 10 transmits the physician's appointment schedule to the terminal apparatus 20.

Step S16: The server 10 receives a desired consultation date and time from the user, and based on the physician's appointment schedule and the user's desired consultation date and time, the server 10 reconciles the two to make a consultation appointment with the physician. With this arrangement, a consultation appointment date and time is set for the user. If a consultation is available on the user's desired consultation date and time, the server 10 sets the consultation appointment date and time based on the user's desired consultation date and time. In the case where making an appointment on the user's desired consultation date and time is difficult, the server 10 sets the date and time closest to the user's desired consultation date and time as the appointment date and time based on the physician's appointment schedule.

Step S17: Based on the position of the physician-affiliated hospital of the physician, the user's position (address or position information), the user's consultation appointment date and time, as well as information such as road conditions along the route, the server 10 generates a dispatch schedule of the vehicle 30 so that the user will be on time for the consultation appointment. The dispatch schedule may also include a schedule for taking the user back home from the hospital. Also, in the case where a transportation service for a plurality of users is necessary in a predetermined time period, the dispatch schedule may be set as a schedule for picking up a plurality of users and stopping at a plurality of hospitals. The generated dispatch schedule is transmitted to the terminal apparatus 20 and the vehicle 30.

(Operation of Terminal Apparatus)

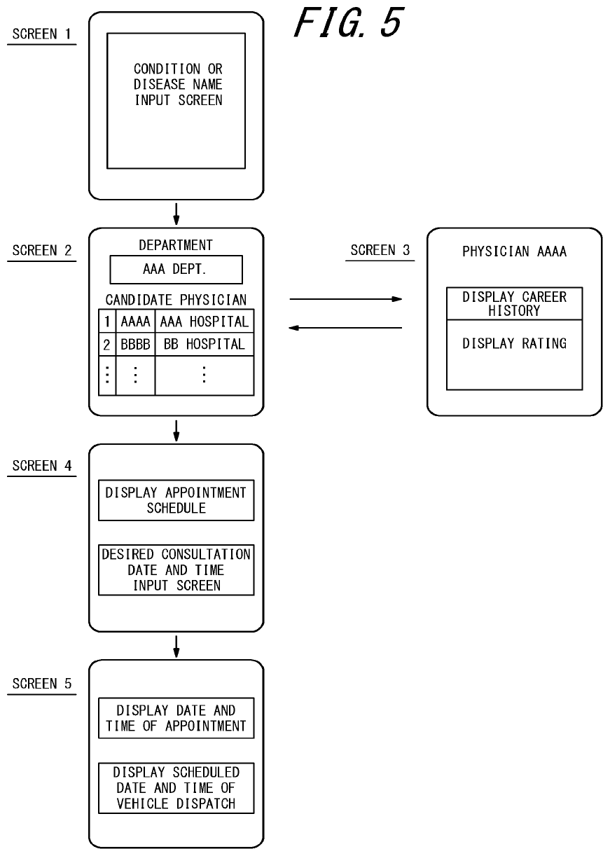

Next, the operation of the terminal apparatus 20 will be described. FIG. 5 is a conceptual diagram illustrating an example of screen transitions on the terminal apparatus 20 of the user. These screens are displayed on the terminal apparatus 20 by downloading a predetermined application program to the terminal apparatus 20 of the user, for example.

Screen 1: First, a screen for inputting a condition or disease is displayed on the terminal apparatus 20. On Screen 1, the user inputs information about his or her current state of health (such as a condition or disease). Although a screen for inputting a condition or disease is displayed as Screen 1 in FIG. 5, initially (before this screen), a screen for inputting personal information about the user to register the user in the system 1 may also be displayed.

Screen 2: On the next Screen 2, the department that the user should visit and the physician(s) providing highly reliable treatment with respect to the user's condition are displayed. On Screen 2, the server 10 presents the results of selecting the optimal physician(s) for the user's state of health based on the input information about the user's state of health. One or multiple candidate physicians may be displayed. Also, in this screen example, only the name of each physician and the name of the physician-affiliated hospital are displayed, but other information may also be displayed. The user selects (chooses) the physician that he or she wants to consult with from among the displayed candidate physician(s). If there is only one candidate physician, or in the case of selecting the top-ranked candidate physician, the user may also make the selection by pressing an approval button.

Also, by selecting a specific candidate physician on this screen, detailed information about the selected candidate physician is displayed. For example, it is possible to transition from Screen 2 to Screen 3.

Screen 3: Screen 3 is a screen displaying detailed information about a selected candidate physician, and for example, the career history and rating of the candidate physician are displayed. The career history and rating may be information registered in the physician database 121 of the server 10. Also, when a candidate physician is selected, the server 10 may extract information such as user rating and review information about the physician from the other database 15, and display the extracted information on the screen. After confirming the displayed information, the user returns to Screen 2.

Screen 4: On Screen 4, the appointment schedule of the selected physician is displayed. Also, an input screen (input area) for inputting a desired consultation date and time is displayed. On this screen, the user inputs a desired consultation date and time, which is transmitted to the server 10. In the case where the appointment schedule of the selected physician is extremely full and setting the desired consultation date and time is difficult, the user can return to Screen 2 and select a different physician.

Screen 5: On the next screen 5, the consultation date and time of the appointment set through the system 1 is displayed. Additionally, the scheduled date and time of the vehicle dispatch set by the server 10 in accordance with the consultation date and time is displayed. Herein, the dispatch date and time when the vehicle 30 is scheduled to arrive at or near the user's home is displayed, but a detailed dispatch schedule (including information such as destinations, a driving route, a scheduled arrival time at each point, and parking times) may also be displayed.

Based on the screen transitions, the user is able to make an appointment for a consultation with a desired physician and also schedule a transportation service.

(Vehicle Configuration)

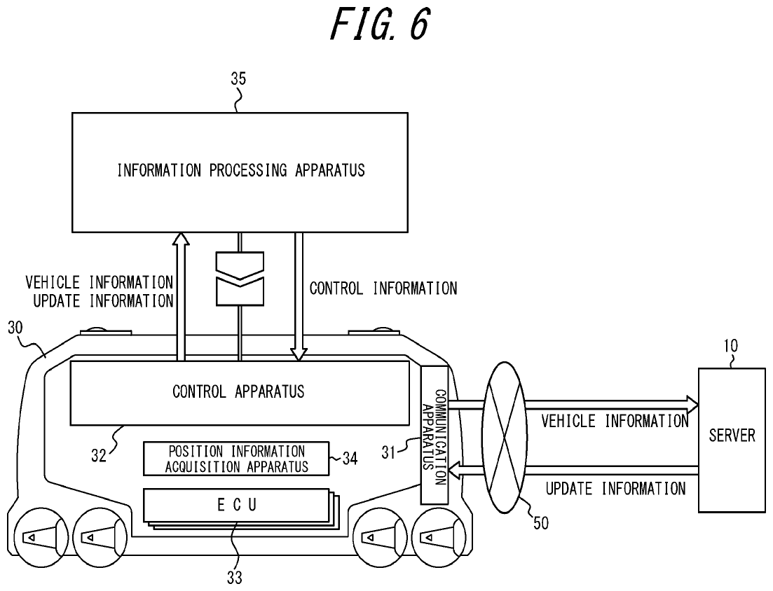

FIG. 6 is a diagram for explaining an exemplary configuration of the vehicle 30 according to an embodiment. The vehicle 30 includes an information processing apparatus 35 configured to control the vehicle 30 as auxiliary equipment. The vehicle 30, the information processing apparatus 35, and the server 10 are connected by the network 50 such as the Internet for example.

The vehicle 30 is for example an automobile capable of autonomous driving or follow-up driving, but is not limited thereto, and may be a vehicle of any type capable of providing a transportation service to the user.

Control of the vehicle 30 is executed through cooperation by the vehicle 30 and the information processing apparatus 35. To summarize, the information processing apparatus 35 automatically generates control information according to autonomous driving control software, and transmits the generated control information to the vehicle 30. The vehicle 30 is controlled based on the received control information. The vehicle control is autonomous driving for example, but is not limited thereto. At least a part of an application programming interface (API) stipulating the specifications of the control information is disclosed to a service provider. The service provider is capable of using the disclosed API to freely program the autonomous driving control software of the information processing apparatus 35. Consequently, by loading equipment according to the purpose into the interior space of the vehicle 30 and programming the autonomous driving control software using the API according to the purpose, the service provider is capable of providing a mobility service of any type.

In the present embodiment, such a vehicle 30 is used in a transportation service to take the user to a hospital, and is capable of autonomous driving from the user's home to the hospital based on a dispatch schedule transmitted from the server 10.

The vehicle 30 includes a communication apparatus 31, a control apparatus 32, a plurality of electronic control units (ECUs) 33, and a position information acquisition apparatus 34. The apparatuses 31 to 34 are communicably connected via an on-board network such as a controller area network (CAN) or dedicated lines, for example.

The communication apparatus 31 may be an on-board communication module such as a data communication module (DCM), for example. The communication apparatus 31 may also include a communication module supporting mobile communications standard such as 4th Generation (4G) and 5th Generation (5G) to connect to the network 50.

The control apparatus 32 is configured to control the vehicle based on the control information from the information processing apparatus 35. The vehicle control is autonomous driving to a destination for example, but is not limited thereto. Autonomous driving includes Levels 1 to 5 of driving automation defined by the Society of Automotive Engineers (SAE) for example, but is not limited thereto, and may be defined in any way. Also, vehicle control is performed through cooperation by components such as the control apparatus 32 and each ECU 33. The control apparatus 32 includes a communication module configured to communicate with the information processing apparatus 35, the communication apparatus 31, and each ECU 33, one or more memories configured to store information such as a system program and an application program, and a controller including one or more processors configured to control the operation of the control apparatus 32 as a whole.

For example, the control apparatus 32 receives various vehicle information related to the vehicle 30 (such as speed, position, and autonomous driving status) from each of the ECUs 33. The control apparatus 32 transmits the vehicle information to the information processing apparatus 35, and also transmits the vehicle information to the server 10 through the communication apparatus 31. In addition, the control apparatus 32 receives update information for the system program and the like of the information processing apparatus 35 from the server 10 through the communication apparatus 31, and transmits the update information to the information processing apparatus 35. Also, in the case of acquiring control information from the information processing apparatus 35, the control apparatus 32 controls the vehicle 30 based on the control information.

The plurality of ECUs 33 cooperate with the control apparatus 32 to control the operation of the vehicle 30. Specifically, the plurality of ECUs 33 receive control instructions based on the control information from the control apparatus 32, and control the operation of the vehicle 30 in accordance with the control instructions. For example, the plurality of ECUs 33 controls operation quantities of the vehicle 30 to be adjusted to the values indicated by the control instructions. Additionally, at each control timing, the plurality of ECUs 33 collect measured values of control quantities or operation quantities of the vehicle 30 from various sensors installed in the vehicle 30, and transmit the measured values to the control apparatus 32.

The position information acquisition apparatus 34 includes one or more receivers to support a satellite positioning system of any type. For example, the position information acquisition apparatus 34 may include a Global Positioning System (GPS) receiver. The position information acquisition apparatus 34 acquires a measured value of the position of the vehicle 30 as position information, and transmits the position information to the control apparatus 32.

The information processing apparatus 35 includes a communication module configured to connect to the control apparatus 32 of the vehicle 30, one or more memories configured to store information such as a system program and an application program, one or more sensors configured to detect information related to the operation of the information processing apparatus 35 or the surrounding environment, and a controller including one or more processors configured to control the operation of the information processing apparatus 35 as a whole. The information processing apparatus 35 is installed in a position such as on the rooftop of the vehicle 30, for example. The position where the information processing apparatus 35 is installed is not limited to the above, and may also be installed at any position of the exterior or interior of the vehicle 30.

The information processing apparatus 35 receives vehicle information and update information from the control apparatus 32. The information processing apparatus 35 also generates control information based on information such as sensor information and vehicle information, and transmits the control information to the control apparatus 32. Additionally, the application program stored in the memory may include autonomous driving control software, and in this case, the information processing apparatus 35 functions as an autonomous driving kit according to the autonomous driving control software.

With this arrangement, the vehicle 30 is capable of performing autonomous driving or follow-up driving.

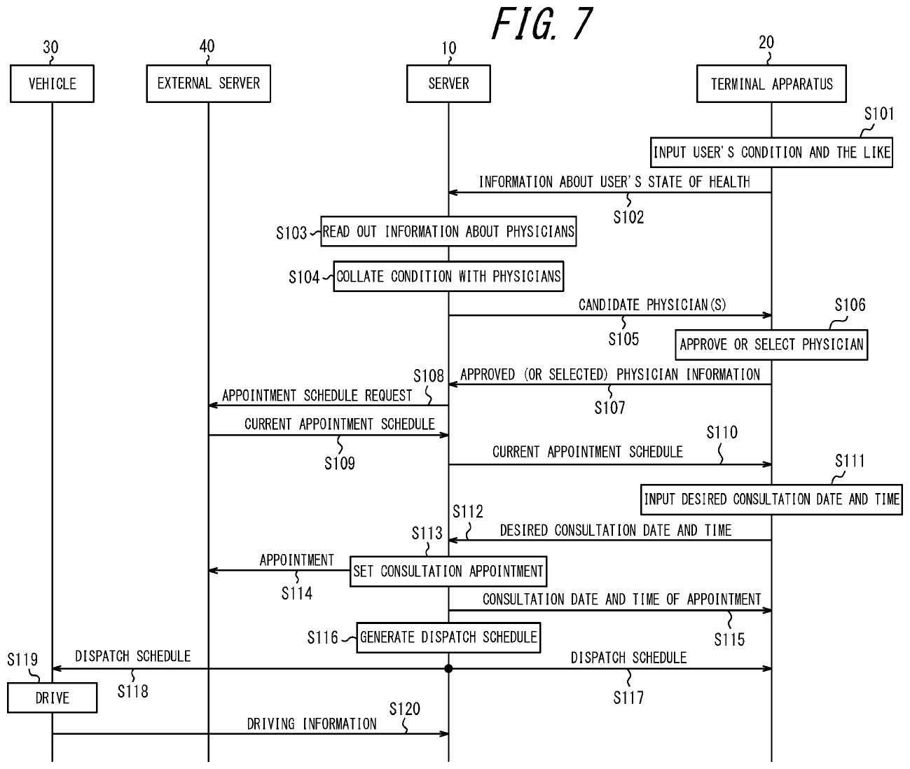

FIG. 7 is a sequence diagram illustrating an example of overall operations by the system 1 according to an embodiment.

Step S101: The terminal apparatus 20 receives information about the user's state of health (such as the condition of the user) as input, and transmits the information to the server 10.

Step S102: The server 10 receives the information about the user's state of health from the terminal apparatus 20. The server 10 may also acquire the information about the user's state of health from a source such as the database 15.

Step S103: The server 10 reads out information about physicians, from the physician database 121 of the server memory 12.

Step S104: The server 10 performs matching by collating the information about the user's state of health (such as the condition of the user) with the information about physicians, and selects an appropriate physician for the user's state of health. For example, the server 10 estimates a disease of the user based on the information stored in the disease name database 122, and additionally selects the physician providing the most highly reliable treatment with respect to the disease and condition of the user based on the information about physicians as a candidate physician. There may also be a plurality of candidate physicians.

Step S105: The server 10 transmits the selected candidate physician(s) to the terminal apparatus 20 of the user. Information related to the candidate physician(s), the inferred disease and department, and the like may also be transmitted at the same time.

Step S106: The terminal apparatus 20 displays the received candidate physician(s), and a candidate physician is approved or selected by the user. When selecting a candidate physician, detailed information about the candidate physician (such as a career history and rating) may also be displayed.

Step S107: Information about the physician that the user has approved or selected is transmitted from the terminal apparatus 20 to the server 10.

Step S108: The server 10 receives the information about the physician that the user has selected, and requests the server (external server 40) owned by the physician-affiliated hospital of the physician to provide information about the appointment schedule of the physician.

Step S109: The hospital server (external server 40) extracts information about the current appointment schedule of the physician, and transmits the extracted information to the server 10.

Step S110: The server 10 forwards the information about the current appointment schedule of the physician received from the hospital server to the terminal apparatus 20.

Step S111: The terminal apparatus 20 displays information about the current appointment schedule of the physician selected by the user, and additionally accepts the input of a desired consultation date and time from the user.

Step S112: The terminal apparatus 20 transmits the desired consultation date and time input by the user to the server 10.

Step S113: The server 10 sets a consultation appointment date and time based on at least one of the user's desired consultation date and time and the physician's appointment schedule. In other words, if an appointment is available, the server 10 sets the consultation appointment date and time based on the user's desired consultation date and time. Also, in the case where making an appointment on the desired consultation date and time is difficult, the server 10 sets the date and time closest to the user's desired consultation date and time as the appointment date and time based on the physician's appointment schedule.

Step S114: The server 10 performs a process of scheduling a consultation appointment with respect to the hospital server (external server 40).

Step S115: The server 10 transmits information about the consultation date and time of the appointment to the terminal apparatus 20.

Step S116: The server 10 generates a dispatch schedule of the vehicle 30 based on the set consultation appointment date and time. Specifically, based on the user's consultation appointment date and time as well as the position of the physician-affiliated hospital of the physician, the user's position (address or position information), as well as necessary information such as road conditions along the route, the server 10 generates a dispatch schedule of the vehicle 30 so that the user will be on time for the consultation appointment.

Step S117: The server 10 transmits the generated dispatch schedule to the terminal apparatus 20. The terminal apparatus 20 displays the dispatch schedule and notifies the user.

Step S118: Also, the server 10 transmits the generated dispatch schedule to the vehicle 30.

Step S119: The vehicle 30 starts driving based on the transmitted dispatch schedule. In other words, the vehicle 30 first heads to the user's home, and after picking up the user, heads to the hospital where the physician is present.

Step S120: While traveling, the vehicle 30 transmits driving information, which includes information such as position information and the traveling speed of the vehicle 30, to the server 10.

According to the present disclosure, it is possible to select a physician appropriate for a disease of the user, and furthermore provide a transportation service to take the user to the hospital where the physician is present.

A computer can be used favorably to function as the server 10 described above. In such a computer, a program stating processing content to achieve each function of the server 10 may be stored in memory of the computer, and the functions can be achieved by having a CPU of the computer read out and execute the program. The program may be recorded onto a computer-readable recording medium.

The foregoing embodiment is described as a representative example, but it should be clear to persons skilled in the art that many alterations and substitutions are possible within the gist and scope of the present disclosure. Consequently, the present disclosure should not be interpreted as being limited by the foregoing embodiment, and various modifications or alterations are possible without departing from the scope of the claims. For example, it is possible to combine a plurality of function blocks described in the embodiment into a single block, or divide a single function block into a plurality of blocks.

1. A server configured to provide a transportation service to take a user to a hospital, comprising:
an acquiring means configured to acquire information about a state of health of the user;
a memory storing information about a physician; and
a controller configured to perform matching by collating the information about the state of health of the user with the information about the physician, sets a consultation date and time with the physician, and generates a dispatch schedule of a vehicle based on the consultation date and time.

2. The server according to claim 1, wherein the acquiring means is a communication interface configured to receive the information about the state of health transmitted from the user.

3. The server according to claim 1, wherein the consultation date and time is decided based on at least one of an appointment schedule of the physician and a desired consultation date and time of the user.

4. A system comprising:
the server according to claim 1;
a terminal apparatus of the user; and
a vehicle configured to provide a transportation service to take the user to a hospital, wherein
the terminal apparatus receives the set consultation date and time and the dispatch schedule, and
the vehicle is driven in accordance with the dispatch schedule.

5. The system according to claim 4, wherein the terminal apparatus transmits, to the server, the information about the state of health of the user.

6. The system according to claim 4, wherein
the server selects, as a candidate physician, a physician providing highly reliable treatment with respect to the state of health of the user and transmits information about the candidate physician to the terminal apparatus, and
the terminal apparatus approves or selects the candidate physician.

7. The system according to claim 4, wherein the terminal apparatus transmits, to the server, the desired consultation date and time of the user.

8. The system according to claim 4, wherein the vehicle performs autonomous driving or follow-up driving.

9. An information processing method for providing a transportation service to take a user to a hospital, comprising:
acquiring information about a state of health of the user;
reading out information about a physician;
performing matching by collating the information about the state of health of the user with the information about the physician, and setting a consultation date and time with the physician; and
generating a dispatch schedule of a vehicle based on the consultation date and time.
